(19) United States
(12) Patent Application Publication
(10) Pub. No.: US 2024/0032506 A1
(43) Pub. Date: Feb. 1, 2024

(54) CLEANING SYSTEM, CONVEYING DEVICE AND SEPARATING DEVICE FOR INSECTS OR WORMS AND CORRESPONDING METHODS

(71) Applicant: BÜHLER INSECT TECHNOLOGY SOLUTIONS AG, Schweiz (CH)

(72) Inventors: Kees Wilhelmus Petrus AARTS, Vught (NL); Maurits Petrus Maria JANSEN, Bavel (NL); Stijn HARMS, Hertogenbosch (NL); Luc DE WOLF, Helmond (NL)

(21) Appl. No.: 18/256,165

(22) PCT Filed: Dec. 10, 2021
(86) PCT No.: PCT/EP2021/085211

§ 371 Date: Jun. 6, 2023

(30) Foreign Application Priority Data
Dec. 11, 2020 (EP) 20 213 352.6

(51) Int. Cl. A01K 13/00; A01K 67/033

(52) U.S. Cl. CPC A01K 13/001; A01K 67/0332

(57) ABSTRACT

The present disclosure relates to a cleaning system for cleaning living insects, in particular living insect larvae or living worms, comprising a collecting device for receiving and mixing the insects or worms with water to form a mixture, a first conveying device for conveying the mixture out of the collecting device and a first larvae-separating device for separating the insects or worms from the water from the mixture conveyed by the first conveying device. The separated water is available for further use in the cleaning system, in particular the water is at least partially recirculated in an insect facility, in particular in the cleaning system.

The present disclosure addresses the processing or treatment of insect larvae. In particular, the present disclosure comprises a cleaning system for cleaning living insects, in particular living insect larvae or living worms, a method using such a cleaning system, a conveyor and a cleaning device.

The invention relates to the field of obtaining nutrients, feedstuffs, and foodstuffs from insects or worms.

In recent decades, interest in the use of insects and worms as food and feed sources has increased—in particular, in view of the growing world population and the increasing demand for alternative and sustainable protein sources for the livestock industry. Since insects and worms are usually rich in proteins and fats, they have a relatively high nutritional or caloric value and are therefore particularly suitable for human nutrition and livestock breeding.

It is desirable in an economic and ecological sense to be able to breed and process insects and worms on an industrial scale to produce standardized nutrients that can subsequently be used to manufacture food or feed. Gentle handling of the still living insects and the production of safe end products is important during processing.

In order to obtain larvae which are as clean as possible without adhering residues from the breeding process, a washing step with water takes place in the processing chain. A disadvantage of known processes and facilities is that, in the separation of insects and water, screening efficiency is often insufficient. Furthermore, water consumption is often high, and gentle transport of the insects along with efficient washing, i.e. sufficient removal of residues from the breeding process, is not always possible. In conventional facilities, it can also happen that live larvae or worms creep through the screen openings.

It is an object of the present invention to overcome the disadvantages known from the prior art. It is further an object of the present invention to provide a cleaning system for cleaning living insects or worms, along with a corresponding method for cleaning living insects or worms, which ensure a gentle and stress-free treatment or processing of living insects or worms, and include water reuse or preparation. Furthermore, the option of storing living insects or worms in water is intended to decouple the breeding of the insects from further processing, whereby both regions can be operated independently of one another and the flexibility in the system is increased. If “insects” or “larvae” are mentioned below, insects, larvae or worms should also be understood as included without express mention.

These objects are achieved by the features of the independent claims. Further advantageous embodiments result from combinations with the features of the corresponding dependent claims and from the statements in the description and figures.

The present invention relates to a cleaning system for cleaning live insects, in particular live insect larvae, or worms according to claim 1 and to the use thereof according to claim 7. The present invention further relates to a conveying device according to claim 9 and to a larvae separating device according to claim 10. The invention is defined in the independent claims. The dependent claims describe preferred embodiments.

In particular, the present disclosure relates to a cleaning system for cleaning living insects, in particular living insect larvae or living worms, comprising a collecting device for receiving and mixing the insects or worms with water to form a mixture, a first conveying device for conveying the mixture out of the collecting device and a first larvae separating device for separating the insects or worms from the water from the mixture conveyed by the first conveying device. The separated water is available for further use in an insect facility, in particular the cleaning system, and in particular is at least partially recirculated in the cleaning system.

Various embodiments may further comprise the following features.

The cleaning system further preferably has at least one storage tank for mass storage of the mixture, wherein the at least one storage tank is preferably arranged downstream from the first conveying device, wherein the cleaning system preferably further comprises at least one additional conveying device for conveying the mixture. The at least one additional conveying device is preferably arranged between the first conveying device and the storage tank or downstream from the storage tank.

Furthermore, another collecting device can be provided for receiving and mixing the insects or the worms with water to form a mixture, wherein the at least one additional collecting device is preferably arranged downstream from the first larvae separating device.

The cleaning system further preferably has at least one additional larvae separating device for further separation of the insects or the worms from the water from the conveyed mixture. Preferably, the separated water is available for further use in the insect facility, in particular in the cleaning system, in particular is at least partially recirculated in the cleaning system. The at least one additional larvae separating device is preferably arranged downstream from the first larvae separating device.

At least one additional water supply for rewatering the mixture can be provided, which preferably opens after the first larvae separating device into the circuit, and/or into the additional collecting device, and/or into the at least one storage tank.

Preferably, at least one water tank is provided for storing water from and for dispensing water into the circuit.

Preferably, at least one filter device is provided for filtering the separated water from the first larvae separating device and/or the at least one additional larvae separating device.

Preferably, at least one temperature control device is provided for controlling the temperature of the water, wherein the at least one temperature control device is preferably arranged between the at least one water tank and the collecting device, or on the water tank.

Preferably, at least one weighing device is provided for determining the weight of the insects or worms, wherein the weighing device is preferably arranged in front of the collecting device or in the collecting device.

In addition, the disclosure comprises a method for cleaning living insects, in particular living insect larvae or living worms with a cleaning system as described above. The method comprises the steps of:
supplying the living insects or living worms into the collecting device;
first mixing the living insects or living worms with water until a particular mixture is achieved;
conveying the mixture to the first larvae separating device by means of the first conveying device;
first separating the mixture in the first larvae separating device;
at least partially providing or recirculating the separated water for further use in the insect facility, in particular the cleaning system.

Various embodiments may further comprise the following features.

The method also preferably comprises conveying the mixture separated in the first larvae separating device to the at least one storage tank by means of the second conveying device and storing the mixture in the at least one storage tank.

The disclosure also comprises a conveying device for conveying living insects, in particular living insect larvae or living worms, in particular in a cleaning system as described above, wherein the conveying device is designed as a contact-free pump, wherein the contact-free pump is in particular a peristaltic pump.

A larvae separating device for separating living insects, in particular living insect larvae or living worms from water, in particular in a cleaning system as described above, according to the present disclosure has a screen drum with a shell section which has meshes with a mesh width of 1.0 mm to 2.5 mm, preferably 1.3 mm to 2.0 mm, and with a transport screw within the screen drum for transporting the insects or the worms from an entry end of the screen drum to an exit end of the screen drum.

Various embodiments may further comprise the following features.

The shell section of the screen drum is preferably a wedge wire sieve. The screen drum preferably has an internal diameter of 500 mm to 3,000 mm, preferably 1,000 mm to 1,500 mm, particularly preferably 1,100 mm to 1,250 mm.

The transport screw preferably has a screw length which extends over the entire length of the screen drum. The transport screw preferably has a screw height of 5 mm to 300 mm, preferably 50 mm to 150 mm, particularly preferably 70 mm to 100 mm. The transport screw preferably has a pitch of 200 mm to 600 mm, preferably 300 mm to 400 mm, particularly preferably 330 mm to 375 mm.

Preferably, at least one spray bar is provided for spraying a liquid by means of preferably a plurality of nozzles. The at least one spray bar preferably has 5 to 40 nozzles, preferably 8 to 28 nozzles. Preferably, the at least one spray bar is arranged within the screen drum and/or outside the screen drum.

Preferably, at least one cleaning element is provided to clean the shell section of the screen drum. Preferably, the at least one cleaning element is designed to clean the outer side of the screen drum and/or is designed as a brush element.

The at least one cleaning element preferably extends over the entire length of the screen drum.

A method for separating living insects, in particular living insect larvae or living worms from water by means of a larvae separating device as described above, according to the present disclosure comprises the steps of:
supplying a mixture of insects, in particular insect larvae, or worms and water, wherein the mixture has a temperature of 5° C. to 30° C., preferably of 8° C. to 15° C., or 20° C. to 26° C., wherein said supply is preferably continuous,
rotating the screen drum, wherein the mixture is dewatered, and the insects or the worms are transported to the exit opening.

Various embodiments may further comprise the following features.

The screen drum preferably has a rotational speed of 2 to 60 revolutions per minute, preferably 5 to 20 revolutions per minute, particularly preferably a rotational speed in the range of 10 to 15 revolutions per minute.

While being transported in the screen drum, the living insects or the living worms are preferably sprayed with water from the at least one spray bar, preferably from at least one spray bar arranged on the inside of the screen drum, wherein the spraying is preferably continuous and/or with a water pressure of 2 bar to 10 bar, preferably of 3 bar to 7 bar. To clean the screen drum, it is preferably sprayed by the at least one spray bar, preferably with a liquid by a spray bar arranged on the outside of the screen drum during the rotation of the screen drum. Preferably, to clean the screen drum, it is contacted by the at least one cleaning element during the rotation of the screen drum.

The invention is explained in more detail below with reference to exemplary embodiments and drawings. In the drawings:

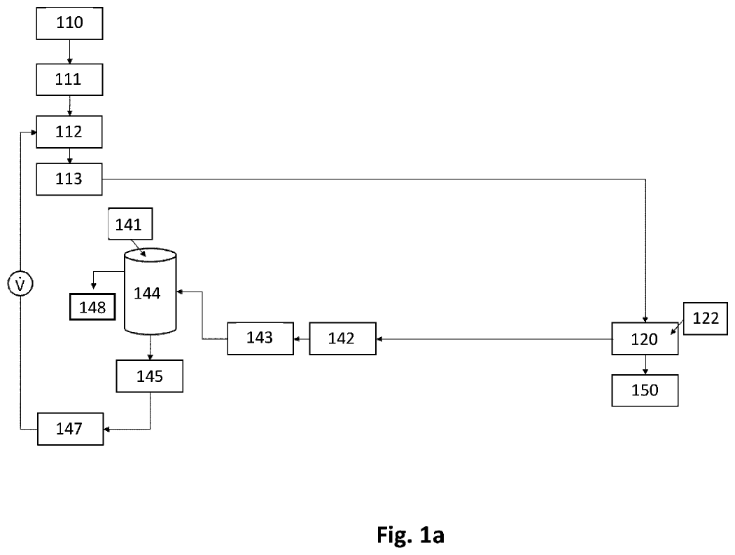

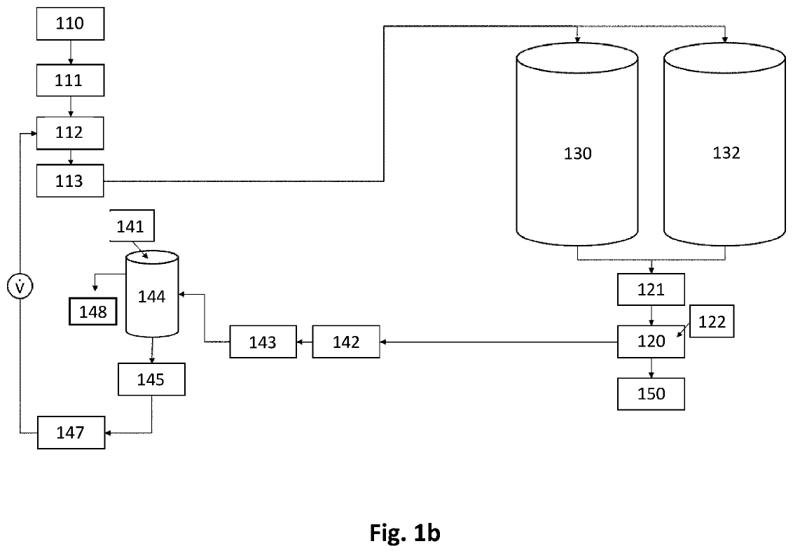

FIG. 1a: shows a schematic illustration of an exemplary embodiment of a cleaning system according to the invention with a cleaning step;

FIG. 1b: shows a schematic illustration of an exemplary embodiment of a cleaning system according to the invention with a cleaning step and the storage of insects or worms;

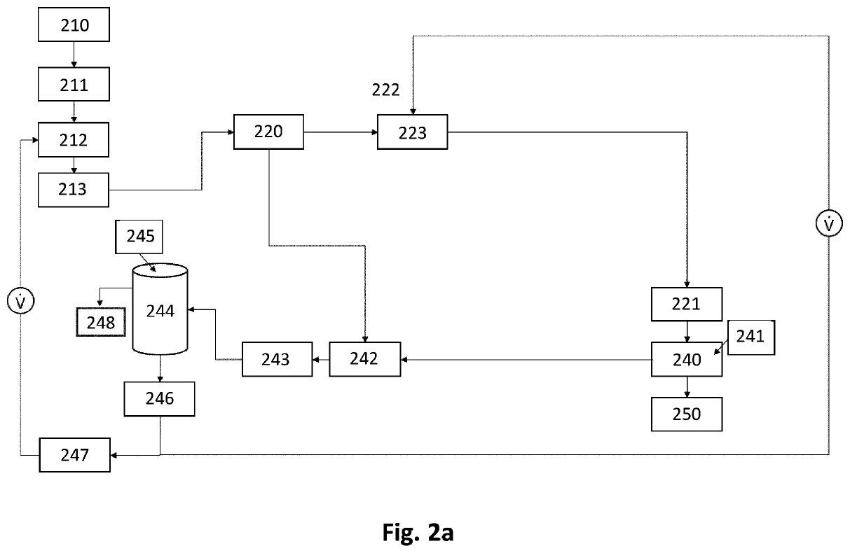

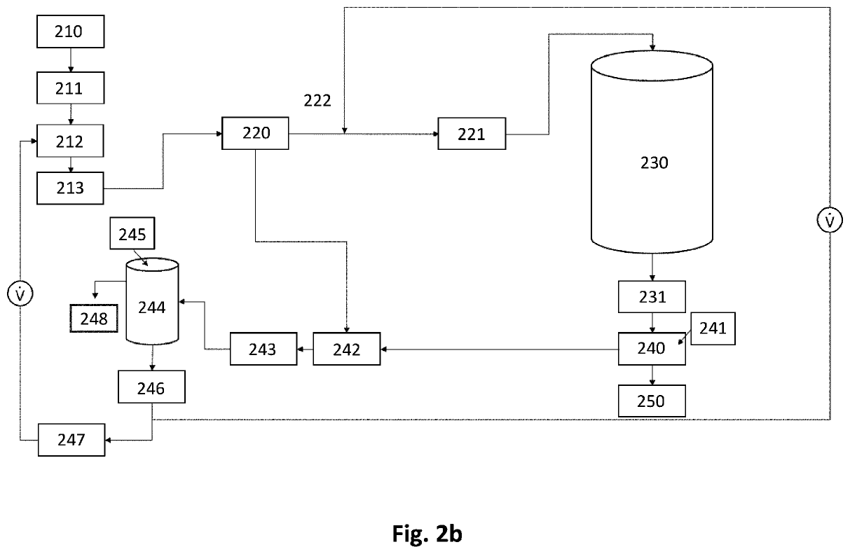

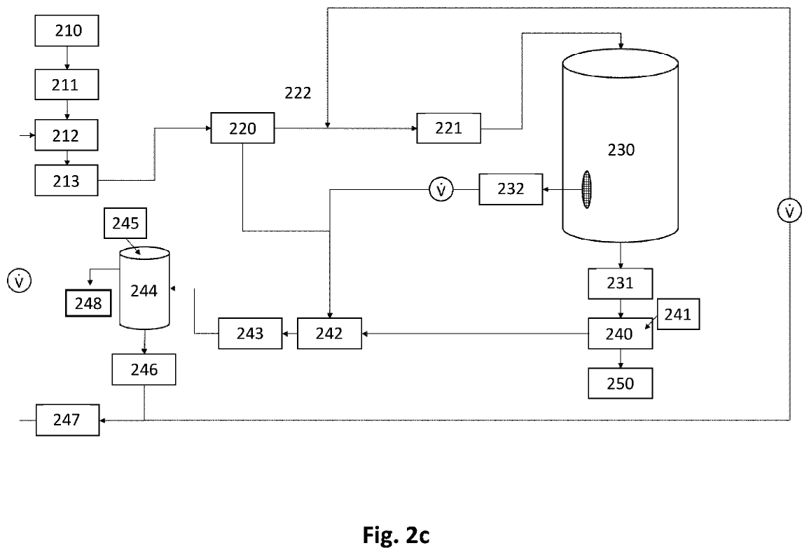

FIG. 2a: shows a schematic illustration of an exemplary embodiment of a cleaning system according to the invention with two cleaning steps;

FIG. 2b: shows a schematic illustration of an exemplary embodiment of a cleaning system according to the invention with two cleaning steps and the storage of insects or worms;

FIG. 2c: shows a schematic illustration of an exemplary embodiment of a cleaning system according to the invention with two cleaning steps, and the storage of insects or worms with a ratio or concentration control in the storage tank;

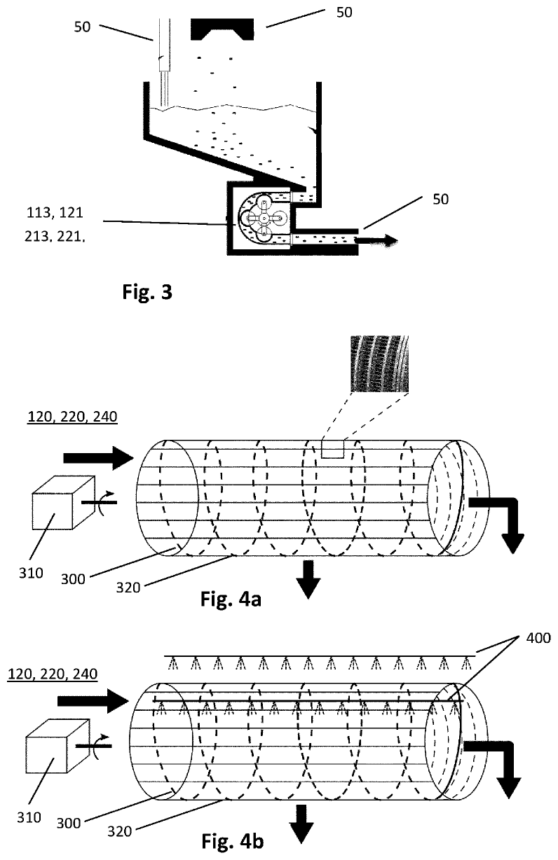

FIG. 3: shows a conveying device according to the invention in a sectional view; and

FIGS. 4a) and 4b): show schematic representations of a larvae separating device according to the invention.

In the following, the same elements, even if they do bear different reference signs, are similar or identical in design unless otherwise specified. The same applies to reference signs. All embodiments can equally be used for insects, in particular insect larvae, and worms.

FIG. 1a shows an exemplary system according to the present disclosure. In particular, a system is shown for treating living insects, in particular living larvae or living worms, with a cleaning step. The system can also be referred to as a cleaning system. +Within the system, living insects or worms are treated with a closed water circuit, which consists of an insect washing apparatus or insect separation system 120, a cleaning device or filter device for waste water 142 and a water temperature control device 145. The water serves both to transport and clean insects and can also be used to clean certain elements of the system, for example the boxes. Instead of water, it is also possible to use another suitable liquid which allows the insects to move together with the liquid within the system. A controller is provided to monitor and adjust the treatment of the larvae and, for example, the ratios between the water and larvae in the system.

The insects, in particular living insects, larvae or worms, come from breeding 110 onto a weighing device 111. The weighing device 111 can be provided separately or integrated into a transport means such as a conveyor belt or transport belt. Moreover, boxes in which insects are grown can be weighed completely. In the weighing device 111, the harvested insects (directly or indirectly together with the box) are weighed, and it is determined how much water must be added in order to obtain a desired weight ratio of larvae to water, for example of around 1:10. For this purpose, the insects are brought into a collecting device 112, to which fresh water is added by means of a pump until the desired weight ratio is achieved. Alternatively or additionally, the weighing device 111 can also be provided integrated in the collecting device 112, or the collecting device 112 can be arranged on a weighing device 111, for example a weighing cell. The ratio 1:10 is mentioned here merely by way of example, and a different amount of water can be added depending on the type of insect and/or system specifications. Mixing ratios between larvae and water of 1:1 to 1:10 have proven advantageous. The temperature of the water can be controlled beforehand by means of a heat exchanger 145. Cooled water makes it possible to cool the insects or worms and bring them into diapause or dormancy. As a result, the insects are metabolically deactivated and can be stored better. This is achieved at temperatures below 15° C., preferably 8° C. to Warmer water, i.e. heated water or water with an ambient temperature of, for example, 20° C. to 30° C., preferably up to 26° C., can yield better cleaning results depending on the soiling. Furthermore, the energy consumption is reduced, since the water does not have to be cooled or heated. However, the insects or worms can be stored for less time, because they are not put into diapause. The mixture of insects, larvae or worms with water is then pumped into a larvae separating device 120 by means of a first conveying device 113. This separates water from larvae in order to change the ratio of larvae to water in the desired manner and will later be described in more detail by way of example. This process can also be referred to as dewatering. Furthermore, the insects are washed during this process, and excrement along with feed residues can be removed. The larvae separating device 120 can also be referred to as a cleaning device. The wastewater is fed to a substrate filter 142, which filters solids out of the water and is pumped into a water tank 144 by means of a pump 143. A centrifuge can also be used instead of a filter 142.

In order to be able to adjust the weight ratio after the larvae separating device 120, to exchange the water in which the insects or worms were stored, or to achieve a better cleaning result, a fresh water supply 122 is provided. The amount of supplied fresh water can be checked by a flow meter (not shown). In particular, the amount of fresh water can be determined based on volume or weight. The water is recirculated in that the water is cleaned by means of the substrate filter 142 and pumped into the water tank 144 by the pump 143. There, wastewater can be removed and, if appropriate, fresh water can be added from a fresh water supply. The supply of fresh water and the removal of waste water can also be expedient at other points in the system.

As described above, the temperature of the cleaned water from the water tank 144 is controlled by means of a heat exchanger 145 as described above and fed back to the circuit by conveying it into the collecting device 112 by a pump 147 and mixing it there with the insects or worms. A flow meter V can also be provided in order to measure the supplied amount of water. The added amount of water can also be checked by weight. Furthermore, the heat exchanger 145 can also be equipped with heating elements for controlling the temperature of the water. Furthermore, the collecting device 112, the conveying device 113, 121 and/or the separating device 120 can also be temperature-controlled in order to be able to adjust the water temperature.

FIG. 1b shows an exemplary system according to the present disclosure. In addition to the embodiment shown in FIG. 1a, the system also has the option of storing insects or worms. Within the system, living insects or worms are treated with a closed water circuit, which consists of at least one storage tank 130, 132, an insect washing apparatus or insect separation system 120, a cleaning device for waste water 142 and a water temperature control device 145. The water serves both to transport and clean the insects. Instead of water, it is also possible to use another suitable liquid which allows the insects to move together with the liquid within the system. A controller is provided to monitor and adjust the treatment of the larvae and, for example, the ratios between the water and larvae in the system.

The insects, in particular living insects, larvae or worms, come from breeding 110 onto a weighing device 111. The weighing device 111 can be provided separately or integrated into a transport means such as a conveyor belt or transport belt. Moreover, boxes in which insects are grown can be weighed completely. In the weighing device 111, the harvested insects (directly or indirectly together with the box) are weighed, and it is determined how much water must be added in order to obtain a desired weight ratio of larvae to water, for example of around 1:10. For this purpose, the insects are brought into a collecting device 112, to which fresh water is added by means of a pump until the desired weight ratio is achieved. Alternatively or additionally, the weighing device 111 can also be provided in the collecting device 112, or the collecting device 112 can be arranged on a weighing device 111, for example a weighing cell. The ratio 1:10 is mentioned here merely by way of example, and a different amount of water can be added depending on the type of insect and/or system specifications. Mixing ratios between larvae and water of 1:1 to 1:10 have proven advantageous. The temperature of the water can be controlled beforehand by means of a heat exchanger 145. Cooled water makes it possible to cool the insects or worms and bring them into diapause or dormancy. As a result, the insects are metabolically deactivated and can be stored better. This is achieved at temperatures below 15° C., preferably 8° C. to Warmer water, i.e. heated water or water with an ambient temperature of, for example, 20° C. to 30° C., preferably up to 26° C., can yield better cleaning results depending on the soiling. Furthermore, the energy consumption is reduced, since the water does not have to be cooled or heated. However, the insects or worms can be stored for less time, because they are not put into diapause. The mixture of insects and larvae or worms is then transported by means of a first conveying device 113 into the at least one storage tank 130, 132. The first conveying device 113 can in particular be designed as a contact-free pump, for example as a peristaltic pump, in order not to injure or crush the insects. In the storage tanks 130, 132, the metabolically deactivated insects, i.e., insects in diapause, can be stored until further processing.

The cleaned insects are pumped from the one or more storage tanks 130, 132 by means of a second conveying device 121, which is likewise preferably designed as a contact-free pump. The ratio of insects to water here is between 1:1 and 1:10, preferably 1:4.

The larvae separating device 120, which is downstream from the second conveying device 121 and will be described below in more detail later by way of example, separates water from larvae in order to change the ratio of larvae to water in the desired manner. This can also be referred to as dewatering. Furthermore, the insects are washed during this process, and excrement along with feed residues can be removed. The larvae separating device 120 can also be referred to as a cleaning device. The wastewater is fed to a substrate filter 142, which filters solids out of the water and is pumped into a water tank 144 by means of a pump 143. A centrifuge can also be used instead of a filter 142.

In order to be able to adjust the weight ratio after the larvae separating device 120, to exchange the water in which the insects or worms were stored, or to achieve a better cleaning result, a fresh water supply 122 is provided. The amount of supplied fresh water can be checked by a flow meter (not shown). In particular, the amount of fresh water can be determined based on volume or weight. The water is recirculated in that the water is cleaned by means of the substrate filter 142 and pumped into the water tank 144 by the pump 143. There, wastewater can be removed and, if appropriate, fresh water can be added from a fresh water supply. The supply of fresh water and the removal of waste water can also be expedient at other points in the system.

As described above, the cleaned water from the water tank 144 is temperature-controlled by means of a heat exchanger 145 as described above and fed back to the circuit, in that it is conveyed by a pump 147 into the collecting vessel 112 and is mixed there with the insects or worms. A flow meter V can also be provided in order to measure the supplied amount of water. The added amount of water can also be checked by weight. Furthermore, the heat exchanger 145 can also be equipped with heating elements for controlling the temperature of the water. Furthermore, the collecting device 112, the conveying device 113, 121 and/or the separating device 120 can also be temperature-controlled in order to be able to adjust the water temperature.

An efficient water circuit is thus ensured which transports, washes and stores insects gently, and at the same time reduces the amount of wastewater that accrues.

FIG. 2a shows an example of another embodiment of a cleaning system according to the present disclosure with two cleaning steps. Unless otherwise indicated, elements with the same name correspond to the elements of the example in FIGS. 1a and 1b and can be used in both embodiments.

The living insects coming from breeding 210, in particular live larvae or living worms, are weighed in the weighing device 211 and transported into the collecting vessel 212, in which they are mixed with water which is pumped from the water tank 244 by means of the pump 247 and is temper-controlled by means of the heat exchanger 246. The water temperatures can be selected as described in connection with FIGS. 1a and 1b. The weighing can also be performed in the collecting vessel or the collecting device 212, or the collecting device can be positioned on a weighing cell. The heat exchanger 246 can cool the water or be equipped with heating elements to control the temperature of the water. It may also be useful to use water at ambient temperature.

In this case as well, the weight ratio of insects to water is preferably 1:10, but other weight ratios may also be useful. The mixture is pumped from a pump 213, preferably a contact-free pump such as a peristaltic pump to the first larvae separating device 220. There, the insects are separated from the water up to a desired weight ratio, in other words dewatered, wherein the excess water flows into the substrate filter 242, is cleaned and pumped through the pump 243 into the water tank 244 and stored there. The cleaned insects or worms, or the cleaned mixture of water and insects or worms, is transported from the larvae separating device 220 into another collecting device 223. The weight ratio of insects or worms to water can be between 1:1 and 1:10, preferably 1:4, in the collecting device 223 and can be adjusted by the fresh water feed 222. Here as well, a flow meter V or a weight-based sensor can be provided in order to measure the amount of fresh water.

The insect/water mixture is then brought by means of the second conveying device 221, which is likewise preferably designed as a contact-free pump, to a second larvae separating device 240, where insects or worms and water, if appropriate with the supply of fresh water 241, are partially or completely separated from one another. The larvae separating device 240 can be similar or identical to the larvae separating device 220. The separated water from the second larvae separating device 240 is cleaned in the substrate filter 242 and pumped by the pump 243 into the water tank 244. There, waste water 248 can be pumped out depending on the state of the water, and fresh water 245 can be supplied.

As already described above and as indicated in the example according to FIG. 1, the water is recirculated from the water tank 244 and mixed with insects in the collecting vessel 212.

FIG. 2b shows an example of another embodiment of a cleaning system according to the present disclosure with two cleaning steps and the option of storing the insects or worms. Unless otherwise indicated, identical elements correspond to the elements of the example of FIGS. 1a, 1b and 2a, and can be used in both embodiments.

The embodiment corresponds to the first larvae separating device 220 shown in FIG. 2a. In the first larvae separating device 220, the insects are separated from the water up to a desired weight ratio, in other words dewatered, wherein the excess water flows into the substrate filter 242, is cleaned and pumped through the pump 243 into the water tank 244 and stored there. As described above, the weight ratio of insects to water after the first larvae separating device 220 can be between 1:1 and 1:10, preferably 1:4, and can be adjusted by the fresh water feed 222. Here as well, a flow meter V or a weight-based sensor can be provided in order to measure the amount of fresh water.

The insect water mixture is then pumped into a storage tank 230 by means of the second conveying device 221, which is likewise preferably designed as a contact-free pump. There, more fresh water can be supplied as required.

If the insects are to be supplied for further processing 250, they are brought by a third conveying device 231 from the storage tank 230 to a second larvae separating device 240, where insects and water, optionally with the supply of fresh water 241, are partially or completely separated from one another. The larvae separating device 240 is similar or identical to the larvae separating device 220. The separated water from the second larvae separating device 240 is cleaned in the substrate filter 242 and pumped by the pump 243 into the water tank 244. There, waste water 248 can be pumped out depending on the state of the water, and fresh water 245 can be supplied.

As already described above and as indicated in the example according to FIGS. 1a, 1b and 2a, the water is recirculated from the water tank 244 and mixed with insects in the collecting vessel 212.

FIG. 2c shows an example of another embodiment of a cleaning system according to the present disclosure. This embodiment makes it possible to adjust the ratio of water to larvae or worms in the tank, and thereby flexibly adjust the amount of larvae or worms in the storage tank. The embodiment is largely identical to that shown in FIG. 2b. In contrast to the example shown in FIG. 2b, a water pump 232 with a downstream flow meter V or a weight-based sensor is provided here, which can pump out water and thereby control the weight ratio between insects and water in the storage tank 230. Preferably, a screen or a restraining device is installed, which prevents insects from being pumped out with the water by the pump 232.

By means of the system presented with reference to the figures described above, breeding and further processing of the insects are separated from one another in terms of process, which allows greater flexibility in production to be achieved. Furthermore, the insects can be transported, washed and stored without damaging them.

FIG. 3 shows a conveying device 113, 121, 213, 221, 231 which is suitable for conveying a mixture of insects and water as described above. FIG. 3 shows a situation in which insects are mixed by a larvae supply 500 with water which is fed from a water supply 501, and the mixture is conveyed through a conveying device, in particular a contact-free pump or peristaltic pump, to an outlet 502. Instead of water, it is also possible to use another suitable non-viscous liquid which allows the mixture of insects and liquid to be pumped. The proposed peristaltic pump is a type of contactless pump in which the rotor blades do not directly touch the liquid to be pumped.

Alternatively, in all embodiments of the present disclosure, other gentle conveying devices such as screw spindle pumps, rotary lobe pumps, rotary piston pumps, piston pumps, eccentric screw pumps or slow running centrifugal pumps can be used.

The weight ratio of insects to liquid is preferably between 1:10 and 1:1. The pump capacity can be between 5 and 100 m³/h. The size of the insects to be pumped is preferably less than 35 mm.

The conveying device can be used in particular in a system as shown in FIGS. 1 and 2. This allows living insects to be transported reliably and efficiently without damaging them. Furthermore, no unnecessary stress is exerted on the animals. The reduction of stress can also be achieved by cooling the liquid to temperatures below 15° C., preferably 8° C. to 15° C., as a result of which the insects are put into diapause.

FIGS. 4a and 4b show an exemplary larvae separating device 120, 220, 240, which differ only by the additional spray bar(s) 400. The larvae separating device 120, 220, 240 can in particular be designed as a screen drum and preferably as a wedge wire mesh. The larvae separating device 120, 220, 240 is driven, in particular rotated, by a drive 310. Furthermore, a transport screw 300 is arranged inside the drum. The larvae separating device 120, 220, 240 has an inlet side and an outlet side (characterized by the arrows in FIGS. 4a and 4b), wherein the insects are transported gently from the inlet side to the outlet side by means of the transport screw 300 attached in the interior. The transport screw extends over the entire length of the screen drum and has a screw height of 5 mm to 300 mm, preferably 50 mm to 150 mm, particularly preferably mm to 100 mm. The pitch is between 200 mm and 600 mm, preferably 300 mm and 400 mm, particularly preferably 330 mm and 375 mm. On the one hand, excess water or a desired proportion of water can thereby be removed, and on the other hand the insects can be washed without damaging or exerting major stress on them.

In addition, the spray bars 400 shown in FIG. 4b can be provided, which spray water from a plurality of nozzles within the larvae separating device 120, 220, 240, and/or spray water outside the larvae separating device 120, 220, 240. As a result of the additional supply of water in the interior of the larvae separating device 120, 220, 240, the washing process can be improved while the screen drum can be cleaned by supplying water from the outside. A different liquid than water or a mixture of water and another liquid can also be sprayed. Each of the spray bars 400 has 5 to 40 nozzles, preferably 8 to 28 nozzles for the spray bar 400 in the interior and 5 to 40 nozzles for the spray bar 400 outside the screen drum. The water pressure can be between 2 bar and 10 bar, preferably between 3 bar and 7 bar.

Alternatively or additionally, a cleaning element that is designed as a brush, for example, can be provided on the outside of the screen drum.

The geometry of the larvae separating device 120, 220, 240 or the screen drum is preferably adapted to the conveying and washing of insects. This prevents injury to insects, stress and clogging of the screen, and cleaning the screen of residues from the breeding process is simplified.

The larvae separating device 120, 220, 240 preferably has a screen drum with a lateral surface 320 which has meshes with a mesh width of 1.0 to 2.5 mm, preferably of 1.3 to 2.0 mm. In particular, the lateral surface 320 of the screen can have a mesh size of 20 to 70%, preferably of 30 to 50%, of the larvae diameter, wherein a larvae size is assumed of 3 to 5 mm in diameter and 12 to 20 mm in length. Such meshes represent holes or openings in the lateral surface of the screen drum. FIG. 4a shows, by way of example, a detail view of the lateral surface 320 and the meshes described above for FIGS. 4a and 4b.

The screen drum has, for example, an internal diameter of 500 mm to 3,000 mm, preferably 1,000 mm to 1,500 mm, preferably of 1,100 mm to 1,250 mm. The screen drum is generally cylindrical in shape, but can also be tapered conically in one of the two directions.

The rotational speed can be 2 to 60 revolutions/min, preferably 5 to 20 revolutions/min, particularly preferably 10 to 15 revolutions/min.

The larvae separating device according to FIG. 4a or 4b can also be used in combination with the conveying device according to FIG. 3 in a system or a cleaning system according to FIG. 1 or 2.

The collecting devices according to the examples described above can also serve to mix insects or larvae with a liquid, for example water. In addition, the collecting device can also be used for storing the insects. Furthermore, the collecting device can also serve for controlling temperature or weighing.

Since, if a plurality of separating devices are provided, the degree of soiling of the water can be different, a plurality of independent or interconnected water circuits can also be provided. This comprises in particular a plurality of filter systems and/or a plurality of water tanks. The water can also be reused for washing the boxes in the system.

If it is expedient and possible, one or more of the conveying devices can be replaced by a gradient. The insects or worms are therefore not actively conveyed, but are fed to the next process step by gravity.

Furthermore, a screw can be provided as a combined conveying and collecting device. These can have options of controlling the temperature and supplying water.

By cooling the water to below 15° C., preferably 8° C. to 15° C., the insects or worms can already be sedated during the transport or cleaning process. The use of water at ambient temperature, for example 15° C. to 30° C., in particular 20° C. to 26° C., can achieve a better cleaning result depending on the soiling, and is more energy-efficient, since the need for cooling is omitted. However, this may also be associated with more stress for the animals, since they are not or only partially sedated during processing. Furthermore, the storage duration of the animals is shortened, since they are not put into diapause.

The invention has been described with reference to a cleaning system with a washing step, a cleaning system with a washing step and storage, a cleaning system with two washing steps, a cleaning system with two washing steps and storage and a cleaning system with two washing steps and storage with a ratio or concentration control in the storage tank. However, more than two washing or separating steps or further intermediate storage steps can also be provided.

The disclosure also comprises a method for cleaning living insects or worms, preferably with a cleaning system as described above.

Furthermore, the disclosure comprises a conveying device for conveying living insects or worms, which gently pumps the insects or worms together with a carrier fluid without damaging the insects or exerting stress on them.

Furthermore, a larvae separating device for separating living insects or worms from water and a corresponding method are provided, whereby living insects can be transported and washed gently, and the weight ratio of insects to water can also be adjusted.

According to the present disclosure, a cleaning system for cleaning living insects or worms is provided, along with a corresponding method for cleaning living insects or worms, which ensures the gentle and stress-free handling or processing of the insects or worms. The liquid used for transporting and cleaning the insects or worms acts as a buffer, whereby fewer mechanical forces act on the animals. In addition, the animals are prevented from being crushed. They can therefore be transported in a stress-free manner, and uniform quality can be ensured. Therefore, the ratio of insects or worms to transport fluid is important for process management. The transport liquid is cleaned and recirculated completely or mostly, resulting in a closed water circuit. This leads to considerably lower water consumption. Furthermore, the insects are metabolically deactivated by cooling liquid and are put into diapause, which allows them to be stored in a storage tank. By means of the presented system, breeding is separated from the cleaning and storage, whereby both areas can be operated independently of one another, and the flexibility in the system is increased.

Although the invention is illustrated and described in detail by means of figures and the associated description, this illustration and this detailed description are to be understood as illustrative and exemplary, and not as limiting the invention. It is understood that those skilled in the art may make changes and modifications without departing from the scope of the following claims. In particular, the invention also comprises embodiments having any combination of features that are mentioned or shown above with respect to various aspects and/or embodiments.

The invention also includes individual features in figures, even if they are shown there in connection with other features and/or are not mentioned above.

Furthermore, the term “comprise” and derivatives thereof do not exclude other elements or steps. Likewise, the indefinite article “a” or “an” and derivatives thereof do not exclude a plurality. The functions of a plurality of features listed in the claims may be fulfilled by one unit. The terms “substantially,” “around,” “approximately,” and the like in conjunction with a property or a value also define in particular precisely the property or precisely the value. None of the reference signs in the claims are to be understood as limiting the scope of the claims.

### LIST OF REFERENCE SIGNS

110 Harvesting
111 Weighing device
112 Collecting vessel/collecting device
113 First conveying device
120 First larvae separating device
121 Second conveying device
122 Fresh water supply
130 Storage tank
132 Storage tank
141 Fresh water supply
142 Filter device/substrate filter/solids filter
143 Water pump
144 Water tank
145 Heat exchanger
147 Water pump
148 Wastewater
150 Processing
210 Harvesting
211 Weighing device
212 Collecting vessel/collecting device
213 First conveying device
220 First larvae separating device
221 Second conveying device
222 Fresh water supply
223 Collecting vessel/collecting device
230 Storage tank
231 Third conveying device
232 Water pump
240 Second larvae separating device
241 Fresh water supply
242 Filter device/substrate filter/solids filter
243 Water pump
244 Water tank
245 Fresh water supply
246 Heat exchanger
247 Water pump
248 Wastewater
250 Processing
300 Transport screw
310 Drive
320 Lateral surface
400 Spray bar
500 Larvae supply
501 Water supply
502 Outlet
V Flowmeter

1. A cleaning system for cleaning living insects, in particular living insect larvae or living worms, comprising
a collecting device for receiving and mixing the insects or the worms with water to form a mixture;
a first conveying device for conveying the mixture from the collecting device; and,
a first larvae separating device for separating the insects or the worms from the water from the mixture conveyed by the first conveying device, wherein the separated water is available for further use in an insect facility, in particular in the cleaning system, and in particular is at least partially recirculated in the cleaning system.

2. The cleaning system according to claim 1, further comprising:
at least one storage tank for mass storage of the mixture, wherein the at least one storage tank is preferably arranged downstream from the first conveying device, wherein the cleaning system preferably further comprises:
at least one additional conveying device for conveying the mixture,
wherein the at least one additional conveying device is preferably arranged between the first conveying device and the storage tank or downstream from the storage tank.

3. The cleaning system according to claim 1, further comprising:
at least one additional collecting device for receiving and mixing the insects or the worms with water to form a mixture, wherein the at least one additional collecting device is preferably arranged downstream from the first larvae separating device.

4. The cleaning system according to claim 1, further comprising:
at least one additional larvae separating device for further separation of the insects or the worms from the water from the conveyed mixture, wherein preferably the separated water is available for further use in the insect facility, in particular in the cleaning system, in particular at least partially recirculated in the cleaning system,
wherein the at least one additional larvae separating device is preferably arranged downstream from the first larvae separating device.

5. The cleaning system according to claim 1,
wherein at least one additional water supply is provided for the rewatering of the mixture, which preferably opens after the first larvae separating device into the circuit and/or into the additional collecting device and/or into the at least one storage tank, and/or
wherein preferably at least one water tank is provided for storing water and for dispensing water into the circuit, and/or
wherein preferably at least one filter device is provided for filtering the separated water from the first larvae separating device and/or the at least one additional larvae separating device.

6. The cleaning system according to claim 1,
wherein at least one temperature control device is provided for controlling the temperature of the water, wherein the at least one temperature control device is preferably arranged between the at least one water tank and the collecting device, or on the water tank, and/or
wherein the cleaning system further comprises a weighing device for determining the weight of the insects or worms, wherein the weighing device is preferably arranged in front of the collecting device or in the collecting device.

7. A method for cleaning living insects, in particular living insect larvae or living worms with a cleaning system according to claim 1, comprising the steps of:
supplying the living insects or living worms into the collecting device;
first mixing the living insects or living worms with water until a particular mixture is achieved;
conveying the mixture to the first larvae separating device by means of the first conveying device;
first separating the mixture in the first larvae separating device; and,
at least partially providing or recirculating the separated water for further use in the insect facility, in particular in the cleaning system.

8. The method according to claim 7, further comprising
conveying the mixture separated in the first larvae separating device to the at least one storage tank by means of the second conveying device; and,
storing the mixture in the at least one storage tank.

9. A conveying device for conveying living insects, in particular living insect larvae or living worms, in particular in a cleaning system according to claim 1, wherein the conveying device is designed as a contact-free pump, wherein the contact-free pump is in particular a peristaltic pump.

10. A larvae separating device for separating living insects, in particular living insect larvae or living worms from water, in particular in a cleaning system according to claim 1, comprising:
a screen drum having a shell section which has meshes with a mesh width of 1.0 mm to 2.5 mm, preferably 1.3 mm to 2.0 mm; and,
with a transport screw within the screen drum for transporting the insects or the worms from an entry end of the screen drum to an exit end of the screen drum.

11. The larvae separating device according to claim 10,
wherein the shell section of the screen drum is a wedge wire sieve, and
wherein the screen drum preferably has an internal diameter of 500 mm to 3,000 mm, preferably 1,000 mm to 1,500 mm, more preferably of 1,100 mm to 1,250 mm, and/or
wherein the transport screw has a screw length extending over the entire length of the screen drum, and/or
wherein the transport screw has a screw height of 5 mm to 300 mm, preferably 50 mm to 150 mm, particularly preferably 70 mm to 100 mm, and/or
wherein the transport screw has a pitch of 200 mm to 600 mm, preferably 300 mm to 400 mm, particularly preferably 330 mm to 375 mm.

12. The larva separating device according to claim 10,
wherein at least one spray bar is provided for spraying a liquid by means of preferably a plurality of nozzles, wherein
the at least one spray bar has 5 to 40 nozzles, preferably 8 to 28 nozzles,
wherein the at least one spray bar is preferably arranged within the screen drum, and/or
the at least one spray bar is arranged outside the screen drum.

13. The larva separating device according to claim 10,
wherein at least one cleaning element is provided to clean the shell section of the screen drum,
wherein the at least one cleaning element is preferably designed to clean the outer side of the screen drum and/or
wherein the at least one cleaning element is designed as a brush element,
wherein the at least one cleaning element extends preferably over the entire length of the screen drum.

14. A method for separating living insects, in particular living insect larvae or living worms from water by means of a larvae separating device according to claim 10, comprising the steps of:
supplying a mixture of insects, in particular insect larvae, or worms and water, wherein the mixture has a temperature of 5° C. to 30° C., preferably of 8° C. to 15° C., or 20° C. to 26° C., wherein
the supply is preferably continuous; and,
rotating the screen drum, wherein the mixture is dewatered, and the insects or the worms are transported to the exit opening.

15. The method according to claim 14,
wherein the screen drum has a rotational speed of 2 to 60 revolutions per minute, preferably 5 to 20 revolutions per minute, particularly preferably a rotational speed of 10 to 15 revolutions per minute, and/or
wherein the living insects or the living worms are sprayed with water from the at least one spray bar, preferably at least one spray bar arranged on the inside of the screen drum during transport in the screen drum, wherein the spraying is preferably continuous and/or takes place with a water pressure of 2 bar to 10 bar, preferably of 3 bar to 7 bar, and/or
wherein, to clean the screen drum, it is sprayed by the at least one spray bar, preferably with a liquid by a spray bar arranged on the outside of the screen drum during the rotation of the screen drum, and/or
wherein, to clean the screen drum, it is contacted by the at least one cleaning element during the rotation of the screen drum.
